(19) United States
(12) Patent Application Publication
(10) Pub. No.: US 2007/0179507 A1
(43) Pub. Date: Aug. 2, 2007

(54) FISTULA GRAFT DEPLOYMENT SYSTEMS AND METHODS

(76) Inventor: Bhavin Shah, West Lafayette, IN (US)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(21) Appl. No.: 11/669,709

(22) Filed: Jan. 31, 2007

Related U.S. Application Data
(60) Provisional application No. 60/763,550, filed on Jan. 31, 2006.

(51) Int. Cl. A61B 17/26

(52) U.S. Cl. 606/113

(57) ABSTRACT

Described are systems and methods useful for treating fistulae. Certain embodiments of the invention relate to fistula graft deployment systems including: (i) an elongate probing member having a lumen, wherein the probing member includes an end configured to pass through at least a secondary fistula opening and a segment of the fistula tract; (ii) a fistula graft device retaining element extending through the probing member lumen; and (iii) a fistula graft device releasably retained by the retaining element, wherein the fistula graft device includes a biocompatible graft body configured to block at least the primary fistula opening.

### REFERENCE TO RELATED APPLICATION

The present application claims the benefit of U.S. Provisional Patent Application Ser. No. 60/763,550 filed Jan. 31, 2006 entitled FISTULA GRAFT DEPLOYMENT SYSTEMS AND METHODS which is hereby incorporated by reference in its entirety.

### BACKGROUND

The present invention relates generally to medical devices and in particular aspects to systems and methods useful for deploying fistula grafts within patients to treat fistulae including those having a primary opening in the alimentary canal.

As further background, a variety of fistulae can occur in humans. These fistulae can occur for a variety of reasons, such as but not limited to, as a congenital defect, as a result of inflammatory bowel disease, such as Chron's disease, irradiation, trauma, such as childbirth, or as a side effect from a surgical procedure. Further, several different types of fistulae can occur, for example, urethro-vaginal fistulae, vesico-vaginal fistulae, tracheo-esophageal fistulae, gastro-cutaneous fistulae, and any number of anorectal fistulae, such as recto-vaginal fistula, recto-vesical fistulae, recto-urethral fistulae, or recto-prostatic fistulae.

Anorectal fistulae can result from infection in the anal glands, which are located around the circumference of the distal anal canal that forms the anatomic landmark known as the dentate line. Approximately 20-40 such glands are found in humans. Infection in an anal gland can result in an abscess. This abscess then can track through soft tissues (e.g., through or around the sphincter muscles) into the perianal skin, where it drains either spontaneously or surgically. The resulting void through soft tissue is known as a fistula. The internal or inner opening of the fistula, usually located at or near the dentate line, is known as the primary opening. Any external or outer openings, which are usually located in the perianal skin, are known as secondary openings.

The path which these fistulae take, and their complexity, can vary. A fistula may take a take a “straight line” path from the primary to the secondary opening, known as a simple fistula. Alternatively, the fistula may consist of multiple tracts ramifying from the primary opening and have multiple secondary openings. This is known as a complex fistula.

The anatomic path which such fistulae take is classified according to its relationship to the anal sphincter muscles. The anal sphincter consists of two concentric bands of muscle, the inner or internal sphincter and the outer or external anal sphincter. Fistulae which pass between the two concentric anal sphincters are known as inter-sphincteric fistulae. Those which pass through both internal and external sphincters are known as trans-sphincteric fistulae, and those which pass above both sphincters are called supra-sphincteric fistula. Fistulae resulting from Crohn's disease usually “ignore” these anatomic planes, and are known a “extra-anatomic” fistulae.

Many complex fistulae consist of multiple tracts, some blind-ending and others leading to multiple secondary openings. One of the most common complex fistulae is known as a horseshoe fistula. In this instance, the infection starts in the anal gland (primary opening) at or near the 12 o'clock location (with the patient in the prone position). From this primary opening, fistulae pass bilaterally around the anal canal, in a circumferential manner. Multiple secondary openings from a horseshoe fistula may occur anywhere around the periphery of the anal canal, resulting in a fistula tract with a characteristic horseshoe configuration.

One technique for treating a perianal fistulae is to make an incision adjacent the anus until the incision contacts the fistula and then excise the fistula from the anal tissue. This surgical procedure tends to sever the fibers of the anal sphincter, and may cause incontinence.

Other surgical treatment of fistulae involve passing a fistula probe through the tract of the fistula in a blind manner, using primarily only tactile sensation and experience to guide the probe. Having passed the probe through the fistula tract, the overlying tissue is surgically divided. This is known as a fistulotomy. Since a variable amount of sphincter muscle is divided during the procedure, fistulotomy also may result in impaired sphincter control, and even frank incontinence.

Still other methods involve injecting sclerosant or sealant (e.g., collagen or fibrin glue) into the tract of the fistula to block the fistula. Closure of a fistula using a sealant is typically performed as a two-stage procedure, including a first-stage seton placement and injection of the fibrin glue several weeks later. This allows residual infection to resolve and to allow the fistula tract to “mature” prior to injecting a sealant. If sealant or sclerosant were injected as a one-stage procedure, into an “unprepared” or infected fistula, this may cause a flare-up of the infection and even further abscess formation.

There remain needs for improved and/or alternative medical systems and methods that are useful for deploying fistula grafts within patients. The present invention is addressed to those needs.

### SUMMARY

The present invention provides, in certain aspects, unique systems and methods for deploying fistula grafts within patients to treat fistulae, for example, fistulae having at least a primary opening in the alimentary canal, a fistula tract, and a secondary opening. Certain embodiments of the invention relate to fistula graft deployment systems that include a probing member exhibiting suitable characteristics for translation through a fistula, the probing member being associated with a mechanism for securing a material sufficiently thereto for drawing the material into a primary fistula opening. For example, some inventive deployment systems include: (i) an elongate probing member having a lumen, wherein the probing member includes an end configured to pass through at least the secondary opening and a segment of the fistula tract; (ii) a fistula graft device retaining element extending through the probing member lumen; and (iii) a fistula graft device releasably retained by the retaining element, wherein the fistula graft device includes a biocompatible graft body configured to block at least the primary opening of the fistula. The graft body can include any suitable biocompatible material, and preferably comprises a remodelable material, for example, a remodelable extracellular matrix material such as submucosa.

In one particular embodiment, the present invention provides a method of deploying a fistula graft within a patient to treat a fistula having at least a primary opening in the alimentary canal, a fistula tract, and a secondary opening. This method comprises providing a fistula graft deployment system. Included in the system is an elongate probing member having a lumen, wherein a portion of the probing member is positioned within the fistula tract during certain portions of the deployment method. Also included in the deployment system is a fistula graft device retaining element, which extends through the lumen of the probing member. Further included in the deployment system is a fistula graft device releasably retained by the retaining element. The fistula graft device includes a biocompatible graft body. Once a suitable fistula graft deployment system has been provided as described above, this method further comprises manipulating the deployment system so as to lodge the graft body within the primary opening, and releasing the fistula graft device from the retaining element.

Another embodiment of the present invention provides a medical product useful to treat a fistula having at least a primary opening in the alimentary canal, a fistula tract, and a secondary opening. This medical product comprises (a) an elongate probing member having a lumen, wherein the probing member includes an end configured to pass through at least the secondary opening and a segment of the fistula tract; (b) a fistula graft device retaining element extending through the probing member lumen; (c) a fistula graft device releasably retainable by the retaining element, wherein the fistula graft device includes a biocompatible graft body configured to block at least the primary opening of the fistula; and (d) a sealed package enclosing the elongate probing member, the fistula graft device retaining element, and the fistula graft device. In certain aspects, the sealed package includes indicia identifying the contents of the package for use in treating a fistula.

Other objects, embodiments, forms, features, advantages, aspects, and benefits of the present invention shall become apparent from the detailed description and drawings included herein.

### BRIEF DESCRIPTION OF THE DRAWINGS

FIG. 1A is a side view of an illustrative fistula graft deployment system of the invention at one stage of an illustrative deployment procedure.

FIG. 1B is a side view of the fistula graft deployment system of FIG. 1A at another stage of an illustrative deployment procedure.

FIG. 1C is a side view of the fistula graft deployment system of FIG. 1A at still another stage of an illustrative deployment procedure.

FIG. 2 provides a side view of an illustrative fistula graft delivery device of the invention.

FIG. 3 provides a top view of a medical product of the invention.

### DETAILED DESCRIPTION

While the present invention may be embodied in many different forms, for the purpose of promoting an understanding of the principles of the present invention, reference will now be made to the embodiments illustrated in the drawings, and specific language will be used to describe the same. It will nevertheless be understood that no limitation of the scope of the invention is thereby intended. Any alterations and further modifications in the described embodiments and any further applications of the principles of the present invention as described herein are contemplated as would normally occur to one skilled in the art to which the invention relates.

As disclosed above, in certain aspects, the present invention provides unique systems for deploying fistula grafts within patients to treat fistulae having at least a primary opening in the alimentary canal, a fistula tract, and a secondary opening. For example, some fistula graft deployment systems of the invention include: (i) an elongate probing member having a lumen, wherein the probing member includes an end configured to pass through at least the secondary opening and a segment of the fistula tract; (ii) a fistula graft device retaining element extending through the probing member lumen; and (iii) a fistula graft device releasably retained by the retaining element, wherein the fistula graft device includes a biocompatible graft body configured to block at least the primary opening of the fistula. The graft body can include any suitable biocompatible material, and preferably comprises a remodelable material, for example, a remodelable extracellular matrix material such as porcine small intestinal submucosa. The invention also provides methods utilizing such fistula graft deployment systems and medical products that include such systems enclosed within sterile packaging.

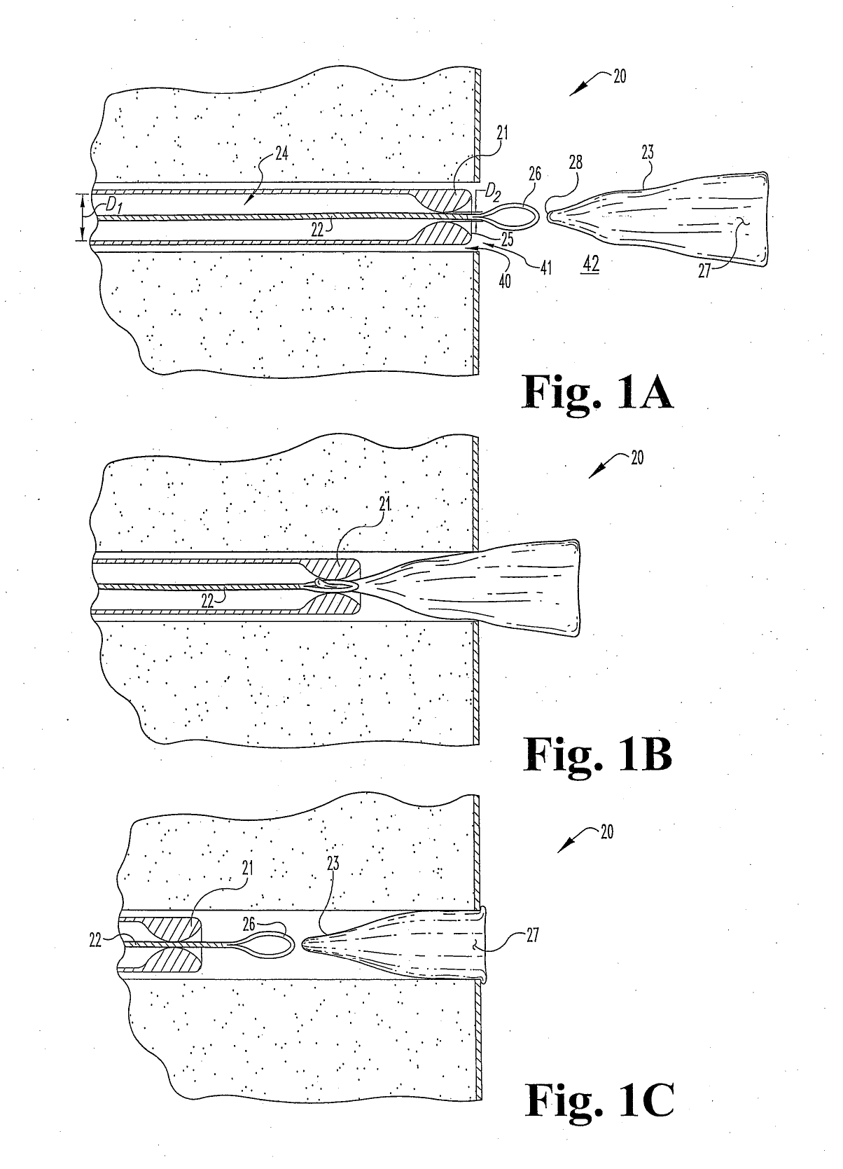

With reference now to FIGS. 1A through 1C, shown are various stages of an illustrative fistula graft deployment procedure utilizing a fistula graft deployment system of the invention. The deployment system 20 includes an elongate probing member 21, a fistula graft retaining element 22, and a fistula graft device 23.

The probing member 21 has a lumen 24, and includes a distal end 25 configured to pass through a secondary fistula opening and through at least a segment of the remaining fistula, e.g., through at least a segment of the fistula tract, and potentially also through the primary opening. The lumen 24 generally exhibits a first diameter D1. However, a portion of the lumen 24 proximate the probing member distal end 25 narrows to a second diameter D2 for reasons discussed more thoroughly below. In this particular embodiment, the probing member distal end 25 is initially passed through a secondary fistula opening (not shown), and advanced through a fistula tract 40 to a point at or near the primary opening 41 (as shown in FIG. 1A). The fistula graft device retaining element 22, which is configured to extend through the probing member lumen 24, is similarly advanced through the fistula tract 40, and can be placed during or after placement of the probing member 21. In the current embodiment, the retaining element 22 is advanced a distance beyond the associated probing member distal end 25 and into the alimentary canal 42.

The retaining element 22 comprises a piece of wire including a deformable wire loop 26 on one end. Such a wire loop 26 can be formed in any suitable manner including but not limited to bending one end of a length of wire in a fashion that forms a loop and coupling this end to another portion of the wire at a point along the length of wire. Such coupling can include any suitable coupling means such as but not limited to welding or otherwise bonding, mechanically fastening, and the like. FIG. 1A shows the wire loop 26 in an “open” configuration. When sufficiently positioned within the probing member lumen 24, the wire loop 26 can be deformed to achieve a “closed” configuration to releasably retain the fistula graft device 23 therein. In the current embodiment, deformation of the wire loop 26 is facilitated by a segment of the probing member 21 proximate its distal end 25, where, again, the inner lumen wall narrows from first diameter D1 to second diameter D2. The second lumen diameter D2 is smaller than the width of the deformable wire loop 26, such that when the loop 26 is forced into second lumen diameter D2, it is contacted by the inner lumen wall and forced to deform to a closed or collapsed configuration.

The fistula graft device 23 includes a biocompatible graft body 27 configured to block at least the primary fistula opening 41. The graft body comprises a remodelable ECM material, for example, porcine SIS. As depicted in FIG. 1A, the graft body 27 can be presented in the alimentary canal 42 so that a tail end 28 of the graft body 27 approaches the wire loop 26. Thereafter, the graft body tail end 28 can be passed through the deformable wire loop 26, and the loop 26 (with the tail end 28 received therethrough) can be passed through the probing member distal end 25 and into the second lumen diameter D2 to cause the wire loop 26 to deform as described above. When sufficiently collapsed, the wire loop 26 impinges the graft body tail end 28 that is received therethrough, and thus, grips the graft body tail end 28 to sufficiently releasably retain the same therein. Then, with the graft body 27 releasably retained by the retaining element 22, the probing member 21 and the retaining element 22 can be moved (in unison) back through the fistula tract 40 and toward the secondary opening so as to lodge the graft body 27 desirably within the primary opening 41. As depicted in FIG. 1C, the wire loop 26 can be “disengaged” from the probing member distal end 25 so that it, again, attains an open configuration, releasing the graft body 27 therefrom. This can be accomplished by holding the wire loop 26 in a fixed position, while forcibly moving the probing member distal end 25 away from the primary opening 41, or alternatively, by holding the probing member 21 in a fixed position, while forcibly moving the wire loop 26 toward the primary opening 41. The probing member 21 and fistula graft device retaining element 22 can then be withdrawn from the fistula through the secondary opening.

Turning now to a general discussion of fistula graft deployment systems and methods of the invention useful for deploying fistula grafts within patients. Certain probing members of the invention have a lumen, and include a “leading” distal end configured to pass through a secondary fistula opening and through at least a segment of the remaining fistula. Although not necessary to broader aspects of the invention, this distal end, or any portion thereof, may be tapered to facilitate passage through a secondary fistula opening and other segments of a fistula. Illustratively, such probing members can be passed through a secondary fistula opening, and advanced to any point within a fistula tract, for example, to a point at or near the primary opening as shown in FIG. 1A, or alternatively, through the primary opening and a distance into the alimentary canal. Accordingly, probing members of the invention can exhibit any suitable size and shape so as to be able to perform these functions while avoiding substantially cutting or tearing the surrounding soft tissues. In certain embodiments, the length of a probing member is typically from about 2 inches to about 12 inches, more typically from about 3 inches to about 9 inches, and even more typically from about 4 to about 8 inches. The outside diameter of a probing member is typically from about 0.3 mm to about 3.2 mm, more typically from about 0.5 to about 3.0 mm, and even more typically from about 1.0 mm to about 2.5 mm.

In some forms of the invention, the probing member is configured to be generally straight in its relaxed condition. Such a probing member can be used, in certain aspects, to treat simple or straight fistulae. Alternatively, probing members of the invention can be configured to include one or more portions that are curvilinear, bent, or otherwise suitably shaped. In certain aspects, the distal end of the probing member is curved to a degree to allow for easier passage of the distal end through a complex fistula, e.g., a horseshoe fistula, and/or through the primary fistula opening and into the alimentary canal.

Further in this regard, probing members of the invention can be formed with any suitable material for facilitating deployment of a fistula graft in accordance with the present invention. Such materials may be selected to take advantage of one or more properties of the material such as but not limited to its weight, durability, flexibility, etc. For example, certain advantageous probing members of the invention are formed with materials exhibiting characteristics to enable the probing member to traverse a fistula, or a portion thereof, without buckling or kinking or causing unacceptable damage to soft tissues defining the fistula. Illustratively, the probing member, or selected portions thereof (e.g., the tip of the distal end), can exhibit a degree of flexibility. In this regard, a probing member, or any portion thereof, may be rigid, malleable, semi-flexible, or flexible.

For example, in certain embodiments, a fistula graft deployment system is particularly adapted for treating fistulae that angulate sharply or curve abruptly such as in the case of certain horseshoe fistulae. In some of these embodiments, the probing member is configured to be directable or steerable through the fistula tract, and therefore, exhibits desirable characteristics, e.g., sufficient stiffness, to allow an operator to apply an adequate degree of ante-grade force to the probing member to allow it to traverse the fistula tract without substantially buckling or kinking.

In other embodiments, the probing member is rigid or substantially rigid, and is configured to be generally straight, for example, for use in treating certain simple or straight fistulae. In other aspects of the invention, the probing member is composed of a malleable material such as but not limited to a woven or spirally-configured metal or alloy material, or a plastic (hydrocarbon-based) material, which may be bent to the necessary angle or curvature, to allow passage through the fistula tract. The shape of such a probing member may be adjusted at certain intervals of the procedure so as to allow the probing member to pass further and further into the fistula tract, until the primary opening is identified. In some forms, the probing member is generally straight in a relaxed condition but can flex to adapt to contours during passage.

Suitable materials for forming probing members of the invention can include but are not limited to metallic materials including stainless steel, titanium, cobalt, tantalum, gold, platinum, nickel, iron, copper and the like, as well as alloys of these metals (e.g., cobalt alloys, such as Elgiloy®, a cobalt-chromium-nickel alloy, MP35N, a nickel-cobalt-chromium-molybdenum alloy, and Nitinol®, a nickel-titanium alloy). Additionally or alternatively, the probing member can include material in the form of yarns, fibers, and/or resins, e.g., monofilament yarns, high tenacity polyester, and the like. A probing member can also include other plastic, resin, polymer, woven, and fabric surgical materials, other conventional synthetic surgical materials, such as a shape-memory plastic, and/or combinations of such materials. Further, appropriate ceramics can be used, including, without limitation, hydroxyapatite, alumina and pyrolytic carbon.

The probing member lumen may or may not exhibit a constant diameter along its length. In certain embodiments, for example as shown in FIGS. 1A through 1C, the probing member lumen includes a segment configured to aid or facilitate releasable retention of the fistula graft device by the fistula graft device retaining element (although such probing member “segments” are certainly not necessary to broader aspects of the invention). In this particular embodiment, a portion of the probing member lumen gradually narrows from a first diameter D1 to second diameter D2 in a curvilinear-like fashion. In other embodiments, such segments are configured to perform a similar function (i.e., aid or facilitate releasable retention of the fistula graft device), yet are structured differently than the segment shown in FIGS. 1A through 1C. Illustratively, a portion of the probing member lumen can narrow from a first diameter to second diameter in a generally linear fashion or in another suitable fashion, or additionally or alternatively, inner wall surfaces of the probing member can include protuberances or the like to help releasably grip the graft device. Such alternative probing member segments can be configured in any suitable manner, with their size and shape (including the size and shape of the corresponding lumen) potentially depending on the size and shape of the fistula graft device retaining element as discussed in more detail below.

In some forms, the probing member lumen maintains generally the same diameter along its length. For example, a probing member can be constructed that is similar in all respects to that shown in FIGS. 1A through 1C, except that its lumen has a constant diameter along its length that is equal to the smaller diameter D2. A device including such a probing member could be operated as discussed above to move the deformable loop 26 in and out of the lumen to achieve closed and open configurations, respectively. Alternatively, a probing member can be constructed that is similar in all respects to that shown in FIGS. 1A through 1C, except that its lumen has a constant diameter along its length that is equal to the larger diameter D1 and the retaining element has additional or alternative features that enable it to releasably grip a fistula graft device.

In certain aspects, a fistula treatment method of the invention includes an endoscopic visualization (fistuloscopy) step that is performed prior to implanting a fistula graft. Such endoscopic visualization can be used, for example, to determine the shape and size of a fistula, which in turn can be used to select an appropriately sized and shaped fistula graft device for treating the fistula. Illustratively, a very thin flexible endoscope can be inserted into a secondary opening of the fistula and advanced under direct vision through the fistula tract and out through the primary opening. By performing fistuloscopy of the fistula, the primary opening can be accurately identified. Also, certain fistula treatment methods of the invention include a fistula cleaning step that is performed prior to implanting a fistula graft. For example, an irrigating fluid can be used to remove any inflammatory or necrotic tissue located within the fistula prior to engrafting the graft device. In certain embodiments, one or more antibiotics are applied to the fistula graft device and/or the soft tissues surrounding the fistula as an extra precaution or means of treating any residual infection within the fistula.

In some modes of operation, means for visualizing and/or irrigating a fistula can be received within the probing member lumen. Illustratively, such means, as well as other desirable instruments and/or materials, can be passed into the proximal end of the probing member lumen (or alternatively, can be passed into one or more openings in a sidewall of the probing member), and through at least a portion of the probing member lumen. For example, in certain aspects, a probing member of the invention includes one or more ports in a sidewall thereof, wherein each port can be associated with a corresponding channel that extends from the port toward the distal end of the probing member. In some forms, one or more port and channel combinations are each configured to receive one or more instruments and/or materials therethrough. For example, a port can be configured to receive one or more optical fibers for visualization and/or illumination of the fistula and surrounding soft tissues, for example, fiber-optic bundles including a plurality of glass fibers comprised of silicone, silicone dioxide, and/or a suitable equivalent. When used in the invention, these optical fibers are provided having suitable characteristics for the particular application including but not limited to suitable lengths and diameters, as well as degrees of flexibility or malleability. Suitable probing member ports can also be configured to receive fluids for the ante-grade irrigation of a fistula. Such fluids can be provided from an external bag of fluid that is connected to the port of the irrigation channel by means of flexible tubing. If necessary, the fluid can be infused under pressure using a pressure bag applied to the fluid source, to increase the pressure under which the fluid is infused. Suitable probing member ports can further be configured to receive guide-wires, drains, solutions such as sealants or sclerosants, high intensity light sources, a lever system to steer the probing member (e.g., wherein the probing member and/or its distal tip is directable in one, two, or three planes), and/or any other suitable instruments and/or mateials. In some forms, a probing member port is configured to receive an optical viewing and lens system that may be attached to a video camera, a video monitor, and a video recorder for viewing at the distal end of the probing member.

Fistula graft device retaining elements of the invention can be configured to extend through the lumen of the probing member, and in this regard, can exhibit any suitable size and shape to be able to do so. Further in this regard, any suitable material can be used in forming a retaining element of the invention including any of those previously described for the probing member. Illustratively, the probing member and the fistula graft device retaining element can include one or more of the same materials and/or one or more different materials. As one non-limiting example, the probing member can include a first material, and the retaining element can include a second material, wherein the second material is relatively more rigid than the first material. The relatively less rigid material may be useful, for example, to allow the probing member to be successfully directed or steered through the fistula tract, while the relatively more rigid material may be useful, for example, to allow adequate force to be applied to the retaining element without causing it to buckle or kink.

During a fistula graft deployment procedure, the retaining element (while surrounded, at least in part, by the probing member) can be passed through a secondary fistula opening, and advanced to any point within a fistula tract, for example, to a point at or near the primary opening, or alternatively, through the primary opening and into the alimentary canal as shown in FIG. 1A. Alternatively, the probing member can be suitably positioned within the patient in a first step, and the retaining element can be suitably positioned within the probing member in a second step.

When extending through the probing member lumen and at least a portion of the fistula, the proximal end of the fistula graft device retaining element can protrude from the secondary opening. In this regard, the proximal end of the retaining element can be manipulated by an operator, e.g., a surgeon, during an illustrative fistula graft deployment procedure of the invention. For example, in some embodiments, such a proximal end is forced toward the secondary opening, causing the distal end of the retaining element to pass through the primary opening and into the alimentary canal. After suitably manipulating the deployment system to releasably retain the fistula graft device with at least the retaining element, the proximal end is forced back away (or otherwise caused to move back away) from the secondary opening to cause the distal end to pass back through the primary opening and into the fistula tract so as to sufficiently implant the graft body within the patient to block at least the primary opening of the fistula.

Further in this regard, the fistula graft device retaining element can be configured in any suitable manner, can exhibit any suitable size and shape, and can include any suitable mechanism and/or material for releasably retaining a fistula graft device in accordance with the present invention. Illustratively, the retaining element can include surfaces, which are configured to contact the fistula graft device in a manner that releasably secures the fistula graft device to the retaining element. A retaining element of the invention can have any suitable number of surfaces that are configured in this manner, and any of these surfaces may or may not be movable relative to another surface. Also, such releasable securement may or may not involve deformation and/or penetration of the fistula graft device, or any portion thereof. In certain aspects, frictional force is sufficient to releasably grip or otherwise secure the fistula graft device between surfaces of the retaining element.

In certain forms, the retaining element includes a first surface and a second surface, wherein the first surface can attain at least two different spatial orientations relative to the second surface for releasably retaining the fistula graft device. These differing orientations can be achieved by movement of the first surface, the second surface, or both. Further, movement of either surface may or may not be facilitated by another object or device. For example, a retaining element can include a spring or a hinged portion to enable movement of one or more of such surfaces relative to another surface.

In some embodiments, the retaining element, or any portion thereof, is translatable along the probing member. In these embodiments, such retaining element translation can promote and/or facilitate movement of one or more retaining element surfaces relative to another surface for releasably retaining the fistula graft device. For example, a retaining element can include two or more arms or other suitable members, which are adapted to move between an “open” configuration and a “closed” configuration when sufficiently moved along the probing member, e.g., slid, twisted, or otherwise suitably moved within and/or around the probing member. Such movement may or may not be facilitated by one or more pivoting adaptations, which can join an arm to one or more other arms and/or to the probing member. In such an open configuration, space is provided between two or more of the surfaces into which the fistula graft device, or a portion thereof, can be placed. In such a closed configuration, this space is closed or reduced to sufficiently contact the graft device to releasably retain the device.

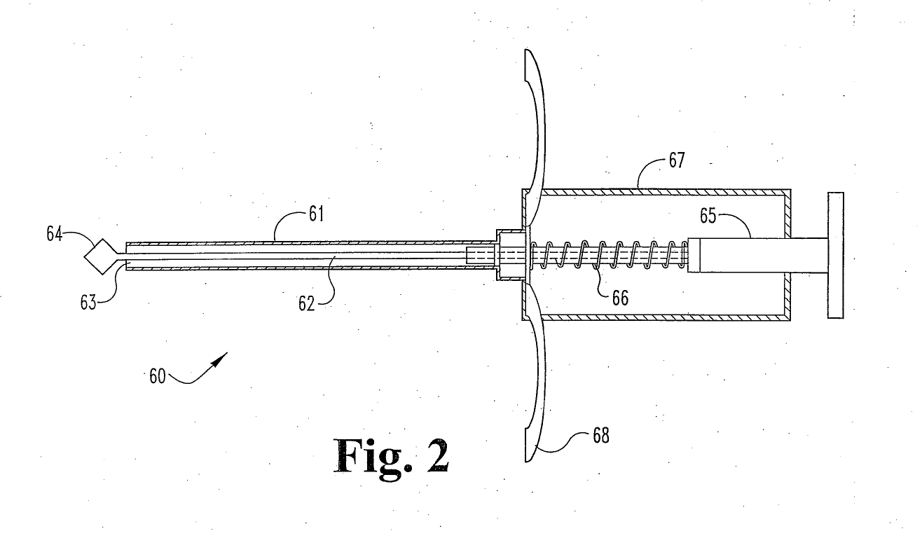

In certain modes of operation, translation of a fistula graft retaining element along (e.g., within the lumen of) a probing member is controlled or otherwise influenced, at least in part, by a spring or spring-like member. For example and referring now to FIG. 2, shown is an illustrative fistula graft delivery device 60 of the invention that includes, inter alia, an elongated, generally cylindrical probing member 61 and a spring-loaded fistula graft retaining element 62 extending through the lumen 63 of the probing member 61.

The distal end of the retaining element 62 includes a deformable wire snare 64. The wire snare 64 is generally in the shape of a diamond, although the snare 64 can exhibit other suitable shapes as well, for example, any of those described elsewhere herein such as that of the deformable wire loop of FIGS. 1A-1C. The proximal end of the retaining element 62 is attached to the distal end of a plunger 65, although in some forms, the plunger 65 is merely an extension of the retaining element 62. Also attached to or otherwise associated with the distal end of the plunger 65 is a spring 66, with a portion of the retaining element 62 including its proximal end extending through the lumen of the spring 66. As depicted, the spring 66 and a portion of the plunger 65 including its distal end are positioned within a generally cylindrical housing member 67. The distal end of the spring 66 is attached to the inside of the housing member 67 proximate its distal end, although such attachment is not necessary to broader aspects of the invention, i.e., the spring 66 need not be connected to any other component of the delivery device 60. The distal end of the housing member 67, which includes an aperture through which portions of the retaining element 62 can pass, is attached to the proximal end of the probing member 61. Nonetheless, it should be noted that certain components of the delivery device 60 could be combined into a single component by one skilled in the art, for example, the probing member 61 and the housing member 67 could form a single component.

Portions of the plunger 65 including its distal end are configured to move back and forth a distance axially within the housing member 67. In this regard, the plunger 65 can be depressed against the resistive force of the spring 66, thereby forcing portions of the retaining element 62 to move distally within the probing member lumen 63. FIG. 2 shows the plunger 65 in a partially depressed position, with the spring 66 in a corresponding partially compressed configuration between the distal end of the plunger 65 and the inside of the distal end of the housing member 67. With the plunger 65 in this position, the snare 64 extends a distance beyond the distal end of the probing member 61 to achieve a relaxed, “open” configuration. Finger grips 68 are attached to the housing member 67 to give the operator leverage in depressing the plunger 65. When the plunger 65 is released, the spring 66 decompresses towards a relaxed configuration, thereby drawing the snare 64 at least partially back into the probing member lumen 63. When sufficiently drawn into the probing member lumen 63, the snare 64 deforms to achieve a “closed” configuration effective to releasably retain a fistula graft device therein. In the current embodiment, the diameter of the probing member lumen 63 is smaller than the width of the deformable wire snare 64, such that when the snare 64 is drawn into the probing member lumen 63, it is contacted by the inner lumen wall and forced to deform to a closed or collapsed configuration.

In use, the distal end of the probing member 61 can be passed through a secondary fistula opening and through at least a segment of the remaining fistula, e.g., through at least a segment of the fistula tract, and potentially also through the primary opening. During such passage, the spring 66 is preferably decompressed so that the deformable wire snare 64, or a substantial portion thereof, is positioned within the probing member lumen 63.

During an illustrative procedure, the distal end of the probing member 61 can be advanced to a point within the fistula tract just shy of the primary opening. Thereafter, the plunger 65 can be depressed with the housing member 67 held in a generally stationary position, forcing the snare 64 out of the probing member lumen 63 and into the alimentary canal. Then, with a portion of a fistula graft device suitably passed through the snare 64 opening, the plunger 65 can be released to cause the snare 64 to pass back into the probing member lumen 63, thereby releasably retaining the graft device in the snare 64. The force of the decompressing spring 66 may be sufficient to draw the graft device into the primary opening to plug it, or alternatively, the delivery device 60 can be retracted a distance through the fistula tract (and potentially back out of the secondary opening depending on the size, shape, and configuration of the fistula graft) to suitably draw the fistula graft into the fistula tract to at least block the primary opening of the fistula or otherwise suitably seat the graft within the fistula tract. Thereafter, the fistula graft device can be suitably separated from the snare 64, preferably by again depressing the plunger 65 to open the snare.

In certain aspects, the retaining element is configured to releasably retain the fistula graft device independent of the probing member. For example, the retaining element can be configured so as to be manually actuatable by the surgeon or other suitable operator to grasp and ungrasp the fistula graft device during a deployment procedure. In other aspects, the retaining element, whether or not coupled to or otherwise joined with the probing member, depends on the probing member to releasably retain the fistula graft device, for example as depicted in FIGS. 1A through 1C.

Suitable fistula graft devices include a biocompatible graft body, which is configured to block at least the primary opening of a fistula, i.e., the primary opening and potentially one or more other segments of a fistula, for example, the fistula tract and/or any secondary openings. In this context, the term “fistula tract” is meant to include, but is not limited to, a void in soft tissues extending from a primary fistula opening, whether blind-ending or leading to one or more secondary fistula openings, for example, to include what are generally described as simple and complex fistulae. As described in more detail below, in certain aspects, fistula graft devices suitable for deployment in accordance with the present invention can also include a suture or other similar adaptation in association with the graft body, which is useful, for example, for forcing the graft body into the primary opening. For example, in certain embodiments, a suture is coupled to the graft body, and the graft device is releasably retained by the retaining element at a point along this suture.

The fistula graft device can be releasably retained by the retaining element, and while being retained, forced into the primary opening so as to lodge the graft body desirably within the primary opening. In certain aspects, forcing the graft body into the primary opening involves pushing the graft body into the primary opening, while in other aspects, forcing the graft body into the primary opening involves pulling the graft body into the primary opening. Upon being suitably lodged within the primary opening, the graft device can be released from the retaining element (either before or after the retaining element, and potentially also the probing member are withdrawn from the fistula through the secondary opening, depending on the characteristics of the particular deployment system being utilized).

The materials used to form the fistula graft devices useful in the invention should generally be biocompatible, and in advantageous embodiments of the devices, use a remodelable material. Particular advantage can be provided by graft devices including a remodelable collagenous material. Such remodelable collagenous materials, whether reconstituted or non-reconstituted, can be provided, for example, by collagenous materials isolated from a warm-blooded vertebrate, and especially a mammal. Such isolated collagenous material can be processed so as to have remodelable properties and promote cellular invasion and ingrowth. Remodelable materials may be used in this context to promote cellular growth on, around, and/or within tissue in which a fistula graft device is implanted, e.g., on, around, and/or within tissue defining a fistula tract or an opening to a fistula. In some forms, the remodelable material can be broken down and replaced by new tissue in such a way that the original fistula closure achieved by the implanted fistula graft is maintained throughout the remodeling process so as to eventually form a closure or substantial closure with the new tissue.

Suitable remodelable materials can be provided by collagenous extracellular matrix (ECM) materials possessing biotropic properties. For example, suitable collagenous materials include ECM materials such as submucosa, renal capsule membrane, dermal collagen, dura mater, pericardium, fascia lata, serosa, peritoneum or basement membrane layers, including liver basement membrane. Suitable submucosa materials for these purposes include, for instance, intestinal submucosa including small intestinal submucosa, stomach submucosa, urinary bladder submucosa, and uterine submucosa. Submucosa when used in the present invention can be obtained by harvesting such tissue sources and delaminating the submucosa from smooth muscle layers, mucosal layers, and/or other layers occurring in the tissue source. For additional information as to submucosa that can be used in the present invention, and its isolation and treatment, reference can be made, for example, to U.S. Pat. Nos. 4,902,508, 5,554,389, 5,993,844, 6,206,931, and 6,099,567.

Submucosa or other ECM tissue when used in the invention is preferably highly purified, for example, as described in U.S. Pat. No. 6,206,931 to Cook et al. Thus, preferred ECM material will exhibit an endotoxin level of less than about 12 endotoxin units (EU) per gram, more preferably less than about 5 EU per gram, and most preferably less than about 1 EU per gram. As additional preferences, the submucosa or other ECM material may have a bioburden of less than about 1 colony forming units (CFU) per gram, more preferably less than about 0.5 CFU per gram. Fungus levels are desirably similarly low, for example less than about 1 CFU per gram, more preferably less than about 0.5 CFU per gram. Nucleic acid levels are preferably less than about 5 μg/mg, more preferably less than about 2 μg/mg, and virus levels are preferably less than about 50 plaque forming units (PFU) per gram, more preferably less than about 5 PFU per gram. These and additional properties of submucosa or other ECM tissue taught in U.S. Pat. No. 6,206,931 may be characteristic of any ECM tissue used in the present invention.

A typical layer thickness for an as-isolated submucosa or other ECM tissue layer useful in some forms of the invention ranges from about 50 to about 250 microns when fully hydrated, more typically from about 50 to about 200 microns when fully hydrated, although isolated layers having other thicknesses may also be obtained and used. These layer thicknesses may vary with the type and age of the animal used as the tissue source. As well, these layer thicknesses may vary with the source of the tissue obtained from the animal source. Further, the submucosa and other ECM tissue materials useful in certain embodiments of the invention can be employed as xenografts (i.e., cross species, such as a non-human donor for a human recipient), allografts (i.e., intraspecies with a donor of the same species as the recipient) and/or autografts (i.e., the donor and the recipient being the same individual).

Suitable ECM materials may include one or more bioactive agents native to the source tissue. For example, a submucosa or other remodelable ECM tissue material used in some forms of the invention may retain one or more growth factors such as but not limited to basic fibroblast growth factor (FGF-2), transforming growth factor beta (TGF-beta), epidermal growth factor (EGF), and/or platelet derived growth factor (PDGF). As well, submucosa or other ECM materials when used in the invention may retain other native bioactive agents such as but not limited to proteins, glycoproteins, proteoglycans, and glycosaminoglycans. For example, suitable graft materials may include heparin, heparin sulfate, hyaluronic acid, fibronectin, cytokines, and the like. Thus, generally speaking, a submucosa or other ECM material may retain one or more bioactive components that induce, directly or indirectly, a cellular response such as a change in cell morphology, proliferation, growth, protein or gene expression.

Submucosa or other ECM materials of the present invention can be derived from any suitable organ or other tissue source, usually sources containing connective tissues. The ECM materials processed for use in the invention will typically include abundant collagen, most commonly being constituted at least about 80% by weight collagen on a dry weight basis. Such naturally-derived ECM materials will for the most part include collagen fibers that are non-randomly oriented, for instance occurring as generally uniaxial or multi-axial but regularly oriented fibers. When processed to retain native bioactive factors, the ECM material can retain these factors interspersed as solids between, upon and/or within the collagen fibers. Particularly desirable naturally-derived ECM materials for use in the invention will include significant amounts of such interspersed, non-collagenous solids that are readily ascertainable under light microscopic examination with appropriate staining. Such non-collagenous solids can constitute a significant percentage of the dry weight of the ECM material in certain inventive embodiments, for example at least about 1%, at least about 3%, and at least about 5% by weight in various embodiments of the invention.

The submucosa or other ECM material used in the present invention may also exhibit an angiogenic character and thus be effective to induce angiogenesis in a host engrafted with the material. In this regard, angiogenesis is the process through which the body makes new blood vessels to generate increased blood supply to tissues. Thus, angiogenic materials, when contacted with host tissues, promote or encourage the formation of new blood vessels into the materials. Methods for measuring in vivo angiogenesis in response to biomaterial implantation have recently been developed. For example, one such method uses a subcutaneous implant model to determine the angiogenic character of a material. See, C. Heeschen et al., Nature Medicine 7 (2001), No. 7, 833-839. When combined with a fluorescence microangiography technique, this model can provide both quantitative and qualitative measures of angiogenesis into biomaterials. C. Johnson et al., Circulation Research 94 (2004), No. 2, 262-268.

Further, in addition or as an alternative to the inclusion of such native bioactive components, non-native bioactive components such as those synthetically produced by recombinant technology or other methods (e.g., genetic material such as DNA), may be incorporated into an ECM material. These non-native bioactive components may be naturally-derived or recombinantly produced proteins that correspond to those natively occurring in an ECM tissue, but perhaps of a different species (e.g., human proteins applied to collagenous ECMs from other animals, such as pigs). These non-native bioactive components may also be drug substances. Illustrative drug substances that may be added to materials include, for example, anti-clotting agents, e.g. heparin, antibiotics, anti-inflammatory agents, thrombus-promoting substances such as blood clotting factors, e.g., thrombin, fibrinogen, and the like, and anti-proliferative agents, e.g. taxol derivatives such as paclitaxel. Such non-native bioactive components can be incorporated into and/or onto a graft material in any suitable manner, for example, by surface treatment (e.g., spraying) and/or impregnation (e.g., soaking), just to name a few. Also, these substances may be applied to the ECM material in a premanufacturing step, immediately prior to the procedure (e.g., by soaking the material in a solution containing a suitable antibiotic such as cefazolin), or during or after engraftment of the material in the patient.

Graft bodies suitable for deployment in accordance with the present invention can be provided in any suitable state including hydrated, partially hydrated, and dried states. Drying a graft body can be accomplished in any suitable manner including but not limited to subjecting the graft body to lyophilization, air dying, vacuum pressing, and other suitable drying conditions known in the art. However, when drying a graft body, it is advantageous to perform drying operations under relatively mild temperature exposure conditions that minimize deleterious effects upon any ECM materials being used, for example native collagen structures and potentially bioactive substances present. Thus, drying operations conducted with no or substantially no duration of exposure to temperatures above human body temperature or slightly higher, say, no higher than about 38° C., will preferably be used in preparing ECM materials useful in some forms of the present invention. These include, for example, vacuum pressing operations at less than about 38° C., forced air drying at less than about 38° C., or either of these processes with no active heating—at about room temperature (about 25° C.) or with cooling. Relatively low temperature conditions also, of course, include lyophilization conditions.

ECM materials when used in the invention may be free of additional, non-native crosslinking, or may contain additional crosslinking. Such additional crosslinking may be achieved by photo-crosslinking techniques, by chemical crosslinkers, or by protein crosslinking induced by dehydration or other means, and may be used to enhance one or more physical, biological and/or other characteristics of an ECM material, and/or to bond two or more pieces of ECM material together. Nonetheless, because certain crosslinking techniques, certain crosslinking agents, and/or certain degrees of crosslinking can destroy the remodelable properties of a remodelable material, where preservation of remodelable properties is desired, any crosslinking of the remodelable ECM material can be performed to an extent or in a fashion that allows the material to retain at least a portion of its remodelable properties.

A deployable fistula graft device, or any component thereof, can have a level or degree of porosity. Remodelable ECM materials having a relatively more open matrix structure (i.e., higher porosity) are capable of exhibiting different material properties than those having a relatively more closed or collapsed matrix structure. For example, an ECM material having a relatively more open matrix structure is generally softer and more readily compliant to an implant site than one having a relatively more closed matrix structure. Also, the rate and amount of tissue growth in and/or around a remodelable material can be influenced by a number of factors, including the amount of open space available in the material's matrix structure for the infusion and support of a patient's tissue-forming components, such as fibroblasts. Therefore, a more open matrix structure can provide for quicker, and potentially more, growth of patient tissue in and/or around the remodelable material, which in turn, can lead to quicker remodeling of the material by patient tissue.

In certain aspects, a fistula graft device includes at least two regions exhibiting differing properties, e.g., differing porosities. Such differing regions can be established in certain locations, for example, locations providing a particular arrangement or pattern on and/or within the remodelable fistula graft, and in some forms, such differing regions are formed by subjecting the fistula graft to a suitable differential drying process. Illustratively, a graft body can be configured so that portions of the graft body adapted to reside in and/or around the primary opening of the fistula occupy a more diminished porosity region, while portions of the graft body adapted to reside within the fistula tract (and potentially one or more secondary openings) occupy a more open porosity region. In this configuration, the diminished matrix region can help isolate the fistula tract from the alimentary canal, thus inhibiting bacteria and other undesirable substances from passing into the alimentary canal from the fistula, while the more open matrix region serves to promote more rapid closure of the fistula with its desirable remodeling properties.

Fistula graft devices for deployment in accordance with the present invention may include biocompatible materials derived from a number of biological polymers, which can be naturally occurring or the product of in vitro fermentation, recombinant genetic engineering, and the like. Purified biological polymers can be appropriately formed into a substrate by techniques such as weaving, knitting, casting, molding, and extrusion. Suitable biological polymers include, without limitation, collagen, elastin, keratin, gelatin, polyamino acids, polysaccharides (e.g., cellulose and starch) and copolymers thereof.

Suitable biocompatible graft materials suitable for deployment in accordance with the present invention can also include a variety of synthetic polymeric materials including but not limited to bioresorbable and/or non-bioresorbable plastics. Bioresorbable, or bioabsorbable polymers that may be used include, but are not limited to, poly(L-lactic acid), polycaprolactone, poly(lactide-co-glycolide), poly(hydroxybutyrate), poly(hydroxybutyrate-co-valerate), polydioxanone, polyorthoester, polyanhydride, poly(glycolic acid), poly(D,L-lactic acid), poly(glycolic acid-co-trimethylene carbonate), polyhydroxyalkanaates, polyphosphoester, polyphosphoester urethane, poly(amino acids), cyanoacrylates, poly(trimethylene carbonate), poly(iminocarbonate), copoly(ether-esters) (e.g., PEO/PLA), polyalkylene oxalates, and polyphosphazenes. These or other bioresorbable materials may be used, for example, where only a temporary blocking or closure function is desired, and/or in combination with non-bioresorbable materials where only a temporary participation by the bioresorable material is desired.

Non-bioresorbable, or biostable polymers that may be used include, but are not limited to, polytetrafluoroethylene (PTFE) (including expanded PTFE), polyethylene terephthalate (PET), polyurethanes, silicones, and polyesters and other polymers such as, but not limited to, polyolefins, polyisobutylene and ethylene-alphaolefin copolymers; acrylic polymers and copolymers, vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins, polycarbonates; polyoxymethylenes; polyimides; polyethers; epoxy resins, polyurethanes; rayon; and rayon-triacetate.

In certain embodiments, the graft body is formed with material having a suitable volumetric shape or space for promoting blockage of at least the primary fistula opening, such as an anorectal fistula opening. Suitable volumetric graft bodies for use in this aspect of the invention can be prepared, for example, as described in U.S. patent application Ser. No. 11/415,403, titled “VOLUMETRIC GRAFTS FOR TREATMENT OF FISTULAE AND RELATED METHODS AND SYSTEMS” (Cook Biotech Incorporated) filed May 1, 2006, which is hereby incorporated by reference in its entirety. Illustratively, such graft bodies can include a layered volumetric graft construct including, for example, a rolled remodelable material that occupies a substantially unitary volume. In some forms, suitable graft bodies are formed by folding or rolling, or otherwise overlaying one or more portions of a biocompatible material, such as a biocompatible sheet material. The overlaid biocompatible sheet material can be compressed and dried or otherwise bonded into a volumetric shape such that a substantially unitary construct is formed. Such a substantially unitary graft body can then be placed in a fistula in a manner such that it blocks at least the primary fistula opening, and potentially blocks at least a portion of the fistula tract and/or any secondary fistula openings. In some forms, a fistula graft device is formed by randomly or regularly packing one or more pieces of single or multilayer ECM sheet material within a mold and thereafter processing the packed material.

Illustrative volumetric graft bodies will be of sufficient size and shape to block at least the primary fistula opening and extend into at least a portion of the fistula tract. Such graft bodies will generally (but not necessarily) be of sufficient dimension to fill the fistula, or a segment thereof, e.g., the primary fistula opening, the fistula tract, and/or any secondary fistula openings, either alone or in combination with other similar or differing devices. In certain embodiments, such graft bodies will have a length of at least about 0.20 cm, and in many situations at least about 1 cm to about 20 cm (approximately 1 to 8 inches). In illustrative embodiments, the fistula graft device will have a length of from about 2 cm to about 5 cm, or alternatively, from about 2 inches to about 4 inches. Additionally, in certain embodiments, graft bodies will have a diameter, which may or may not be constant along their length, of from about 0.1 mm to about 25 mm, or more typically from about 5 mm to about 10 mm. In certain embodiments, a generally conical plug device is tapered along its length so that the end of the plug device configured for placement in and/or around the primary opening has a diameter of about 5 mm to about 10 mm, and the opposite end of the plug device has a diameter of about 0.5 mm to about 3 mm. Such a taper may or may not be continuous along the length of the graft body.

A graft device, or any portion thereof, can include a suitable biocompatible foam or sponge form material. Illustratively, a graft device may comprise a porous, three-dimensionally stable body formed with one or more suitable biocompatible matrix materials. Such biocompatible matrix materials can include naturally-occurring polymers and/or synthetic polymers. More preferred sponge compositions will comprise collagen as a matrix-forming material, either alone or in combination with one or more other matrix forming materials, and particularly preferred sponge compositions will comprise an ECM material such as those discussed elsewhere herein. In general, sponge matrices useful in certain embodiments of the present invention can be formed by providing a liquid solution or suspension of a matrix-forming material, and causing the material to form a porous three-dimensionally stable structure; however, a sponge or foam material can be formed using any suitable formation method, as is known in the art. For additional information concerning foam or sponge form materials that can be useful in certain embodiments of the present invention, reference can be made, for example, to U.S. Pat. App. Pub. No. 2003/0013989.

In some forms, a compact, stabilized sponge construct is highly expansive when wetted, which can desirably enhance the ability of the graft body to block (and to continue blocking) at least the primary opening of a fistula. These compact, stabilized sponge constructs can be useful to allow the graft body to attain a more low-profile configuration for traversing a fistula. For example, an illustrative graft body can include a suitable sponge construct such that in a stabilized, compressed first configuration, the graft body can fit within the distal end of a probing member exhibiting suitable characteristics so as to be able to traverse a fistula. Illustratively, this end of the probing member can be passed into a secondary opening, through a fistula tract, and out of a primary opening into the alimentary canal. Thereafter, the retaining element, which is at least partially received within the probing member lumen, can be used to force the graft body, or a portion thereof, out of the probing member and into the alimentary canal so as to allow the graft body, or a portion thereof, to attain an expanded second configuration. In such an expanded configuration, the graft body, which was previously able to easily traverse the primary fistula opening, is now sized and shaped so as to desirably contact soft tissues surrounding the fistula and lodge within the primary opening to block the primary opening, and potentially other segments of the fistula, when pulled into the fistula by the retaining element. In illustrative procedures, a suitable hydrant, such as saline, may be applied or delivered to the graft body after it is suitably located within a patient to enhance the expansion of the body within the fistula tract and/or a fistula opening. Alternatively, or additionally, a bodily fluid of the patient can sufficiently wet the implanted graft body so as to promote the expansion of the body within the fistula.

In certain aspects, an illustrative graft body includes a compliant sheet form biocompatible material, e.g., multilaminate sheet material comprising one or more layers of material bonded together. Suitable compliant sheet form graft bodies for use in this aspect of the invention can be prepared, for example, as described in U.S. patent application Ser. No. 11/414,682, titled “FISTULA GRAFT WITH DEFORMABLE SHEET-FORM MATERIAL” (Cook Biotech Incorporated) filed Apr. 28, 2006, which is hereby incorporated by reference in its entirety. Such sheet form materials are deformable upon impingement by soft tissue surrounding a fistula (e.g., tissue surrounding the primary fistula opening, the fistula tract, and/or any secondary fistula openings). These deformable materials can include any of the ECM or other biocompatible materials described herein, for example, a multilaminate sheet of remodelable SIS material. The bioactive nature of such materials promotes desirable healing of the fistula, for example, by overcoming the effects of bacteria and other deleterious substances typical to the fistula environment.

Suitable multilaminate materials, whether used in this or other aspects of the invention, can include a plurality of ECM material layers bonded together, a plurality of non-ECM materials bonded together, or a combination of one or more ECM material layers and one or more non-ECM material layers bonded together. Also, an adhesive, glue or other bonding agent may be used in achieving a bond between two segments of ECM material, e.g., between two layers of ECM material. Suitable bonding agents may include, for example, collagen gels or pastes, gelatin, or other agents including reactive monomers or polymers, for example cyanoacrylate adhesives. As well, bonding can be achieved or facilitated between ECM material layers using a suitable crosslinking technique, e.g., using chemical cross-linking agents, such as glutaraldehyde, formaldehyde, epoxides, genipin or derivatives thereof, carbodiimide compounds, polyepoxide compounds, or other similar agents. A combination of one or more of these with dehydration-induced bonding may also be used to bond ECM material layers to one another.

Further, such sheet form graft bodies are sized and shaped so as to be deformable to a three-dimensional volumetric body blocking at least the primary opening, and potentially filling at least a portion of the fistula tract and/or any secondary openings of the fistula. In so doing, advantageous implant materials will also be sufficiently flaccid to avoid substantial cutting or tearing of the surrounding soft tissues. In certain aspects, such a three-dimensional volumetric body, when formed, includes a portion protruding through any secondary openings of the fistula. This extending portion can be used, in certain aspects, to attach the fistula graft device to soft tissues at or near a secondary opening of the fistula as a means of preventing the graft body from reverse migrating undesirably back toward the alimentary canal.

In certain aspects, a sheet form graft body is shaped and sized such that the diameter of the primary opening is less than the width of the sheet so that as the sheet of material is drawn into the fistula tract, it is forced to fold and/or roll over itself one or more times to conform to soft tissues surrounding the fistula, and is gradually “wedged” into the primary opening, and potentially at least a portion of the fistula tract and/or any secondary openings of the fistula, so as to block these spaces when sufficiently pulled therethrough. Such lodging in place may be sufficient to obviate the need for otherwise securing the graft to the soft tissues at or near the primary opening, fistula tract, and/or any secondary openings. Nonetheless, in certain aspects, the graft is further secured to such soft tissues, for example, by suturing. Also, any portion of the graft body can be trimmed, for example, to prevent the engrafted graft body from protruding undesirably from the primary opening and/or any secondary openings of the fistula.

In addition to those described elsewhere herein, a variety of other suitable fistula graft devices can be used in conjunction with the systems and methods of the present invention. Such graft devices can be prepared, for example, as described in U.S. Provisional Application, entitled “FISTULA GRAFTS AND RELATED METHODS AND SYSTEMS FOR TREATING FISTULAE” (Cook Biotech Incorporated) filed on Jan. 31, 2006, which is hereby incorporated by reference in its entirety.

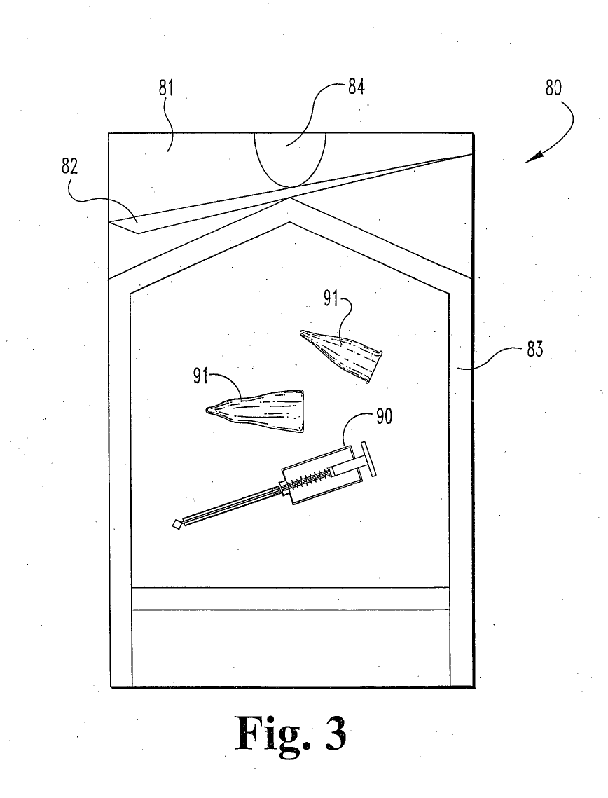

With reference now to FIG. 3, shown is a top view of an illustrative medical product 80 of the present invention that includes a fistula graft delivery device 90 and fistula graft devices 91 sealed within sterile medical packaging. In particular, medical product 80 has packaging including a backing layer 81 and a front film layer 82 (shown partially drawn away from backing layer 81). The fistula graft device is sealed between backing layer 81 and film 82 utilizing a boundary of pressure-adhesive 83 as is conventional in medical packaging. A cut-out 84 may be provided in the backing layer 81 to assist a user in separating the film layer 82 from the backing layer 81.

Sterilization of the medical product 80 may be achieved, for example, by irradiation, ethylene oxide gas, or any other suitable sterilization technique, and the materials and other properties of the medical packaging will be selected accordingly. Also, the fistula graft devices 91 can be contained in sterile packaging in any suitable state. Suitable states include, for example, a hydrated or dehydrated state. The fistula graft devices 91 can be dehydrated by any means known in the art (e.g., lyophilization or air dried). If a fistula graft device is stored in a dehydrated state, it is preferred that it retains all of its biological and mechanical properties (e.g., shape, density, flexibility, etc.) upon rehydration.

The materials and other properties of the packaging will be selected accordingly. For example, the package can include indicia to communicate the contents of the package to a person and/or a machine, computer, or other electronic device. Such indicia may include the dimensions of, the type of materials used to form, and/or the physical state of, the contents of the package. In certain embodiments, a fistula graft device is packaged for sale with instructions for use. For example, in a particularly preferred embodiment, a medical product includes at least one fistula graft device sealed within a sterile package, wherein the packaging has visible indicia identifying the at least one fistula graft device as having physical characteristics as disclosed herein, and/or can contain or otherwise be associated with printed materials identifying the contents as having such physical characteristics and including information concerning its use as a fistula graft device for treating fistulae. The packaging can also include visible indicia relating to the dimension of the at least fistula graft device, and/or relating to the treatment site(s) for which the at least one fistula graft device is configured.

The present invention also provides a line of medical products, wherein a medical product of the invention includes one or more fistula graft devices, one or more probing members, and/or one or more fistula graft device retaining elements such as those disclosed herein enclosed within a sealed package. When the medical product includes more than one of any of these devices, for example, a plurality of fistula graft devices, the devices can each be of substantially the same size and shape, or, alternatively, can vary with respect to size and shape.

A fistula graft device suitable for deployment in accordance with the present invention can be modified before, during, and/or after deployment. Illustratively, a graft body may be cut, trimmed, sterilized, and/or treated (e.g., brought into contact, impregnated, coated, etc.) with one or more desirable compositions, such as any of those previously disclosed herein, e.g., anticoagulants (e.g., heparin), growth factors or other desirable property modifiers. In certain aspects, following deployment of a graft body in accordance with the present invention, one or more portions of the body are trimmed off or otherwise removed, for example, material protruding from the primary opening and/or any secondary opening.

Suitable fistula graft devices can include an anchoring adaptation to maintain the graft body in a desirable location following implantation. For example, an adhesive can be applied to a fistula graft device before an implantation procedure, e.g., during manufacture of the graft device, or alternatively, can be applied to the fistula graft and/or to tissue at or near the primary opening during such an implantation procedure. Other suitable anchoring adaptations include but are not limited to barbs, hooks, sutures, protuberances, ribs, and the like. Again, such anchoring adaptations, while advantageous in certain situations, are not a necessary part of a graft device suitable for deployment in accordance with the present invention. Illustratively, certain fistula graft devices are configured so that the graft is able to maintain a desirable position within the fistula (e.g., blocking at least the primary opening) following implantation without the need for such anchoring adaptations. In other aspects, suitable anchoring adaptations aid or facilitate the maintenance of such a position.

Additionally, in illustrative embodiments, one or more anchors, barbs, ribs, protuberances, and/or other suitable surface modifications can be incorporated on and/or within an illustrative graft body to roughen, condition, or otherwise de-epithelialize at least a portion of the fistula, such as the fistula tract and/or the primary opening, during and/or after emplacement of the graft within the patient. The conditioning of the tissue surrounding the fistula can serve to initiate a localized healing response in patient tissue that can be advantageous in enhancing the ingrowth of patient tissue into an illustrative plug construct, such as a plug comprising an ECM material. Further, in illustrative embodiments, where a graft device include a suture, leader, or string to assist with the emplacement of an illustrative graft construct within a patient, as is discussed elsewhere herein, the leader can comprise an abrasive material, or comprise one or more sections and/or surface features and/or adaptations, e.g. one or more bristles that can directionally emanate from the leader material and that can serve to roughen or otherwise condition or de-epithelialize patient tissue upon travel through and/or location within a fistula.

In certain aspects, fistula graft devices incorporate an adhesive or, where appropriate, a sclerosing agent to facilitate and/or promote blocking of at least the primary opening of the fistula. As well, fistula treatment methods of the invention can include steps where such substances or materials are applied to a fistula graft device being deployed and/or to the soft tissues surrounding the fistula. For example, an adhesive, glue or other bonding agent may also be used in achieving a bond between a fistula graft device and the soft tissues defining a fistula opening or tract. Suitable bonding agents may include, for example, fibrin or collagen gels or pastes, gelatin, or other agents including reactive monomers or polymers, e.g., cyanoacrylate adhesives. In some forms of the invention, a fistula treatment method includes contacting soft tissue surfaces surrounding the fistula, e.g., soft tissue surfaces at or near the primary opening and/or soft tissues lining the fistula tract, with a sclerosing agent prior to forcing the sheet from material into the fistula. Such use of a sclerosing agent can de-epithelialize or otherwise damage or disrupt these soft tissue surfaces, leading to the initiation of a healing response.

As previously mentioned, a fistula graft device can include a suture or other suitable device in association with the graft body. Such a device can be coupled to or otherwise associated with the graft body in any suitable manner, such as but not limited to utilizing a bonding agent or mechanical fastener. In certain aspects, a graft body includes an aperture that extends transversely through the graft, or a portion thereof, which can be used for the receipt of a suture or string. In some forms, the suture is releasably retained by the fistula graft device retaining element, and used to desirably locate the fistula graft within the patient. Thereafter, the suture can be removed from the graft, for example, using cutting shears. In alternative embodiments, the string, suture, etc. can be made from a remodelable or otherwise resorbable material such that the string or suture can be left in place within the fistula tract. In these embodiments, the resorbable or remodelable leader can be used to anchor or otherwise suitably secure the fistula graft within the implantation site. For example, the leader can be tied to patient tissue at a suitable location, for example, a location just inside or external to a secondary fistula opening. Further, in alternative embodiments, an illustrative fistula graft can be positioned so that it spans the entire length of a fistula tract, i.e., from the primary opening to a location at or external to a secondary opening. In these embodiments, the string or suture can be used to secure the tail of the graft to patient tissue at an external location.

Further, any exogenous bioactive substances incorporated into ECM material may be from the same species of animal from which the ECM material was derived (e.g. autologous or allogenic relative to the ECM material) or may be from a different species from the ECM material source (xenogenic relative to the ECM material). In certain embodiments, the ECM material will be xenogenic relative to the patient receiving the graft, and any added exogenous material(s) will be from the same species (e.g. autologous or allogenic) as the patient receiving the graft. Illustratively, human patients may be treated with xenogenic ECM materials (e.g. porcine-, bovine- or ovine-derived) that have been modified with exogenous human material(s) as described herein, those exogenous materials being naturally derived and/or recombinantly produced.

In some embodiments, a fistula is drained prior to receiving a fistula graft device therein. Such draining can be accomplished by inserting a narrow diameter rubber drain known as a seton (Greek, “thread”) through the fistula. The seton is passed through the fistula tract and tied as a loop around the contained tissue and left for several weeks or months, prior to definitive closure or sealing of the fistula. This procedure is usually performed to drain infection from the area, and to mature the fistula tract prior to a definitive closure procedure.

Additionally, fistula graft devices used in conjunction with some of the systems and methods of the present invention include a radiopaque element such as but not limited to a radiopaque coating, attached radiopaque object, or integrated radiopaque substance. These radiopaque elements can allow the movement of the device to be monitored during deployment so that the device may be placed at a desirable location. Any suitable radiopaque substance, including but not limited to, tantalum such as tantalum powder, can be incorporated into a fistula graft device used in the invention. Other radiopaque materials comprise bismuth, iodine, and barium, as well as other suitable markers.

Further, the fistula treatment methods described herein can be used to close one or more fistula during a given medical procedure. Also, the methods of the invention can be used to treat complex fistula. For multiple fistula, multiple fistula graft devices can be engrafted until all the fistula have been addressed. In cases of complex fistula, for example a horse-shoe fistula, there may be one primary opening and two or more fistula tracts extending from that opening. In such instances, a fistula graft device may be configured with a graft body including one “head” and two “tails.” Each tail can be drawn into the primary opening, and thereafter into one of the fistula tracts extending therefrom. Each of the tails can be secured by sutures and/or an adhesive, if necessary, and any excess material can be trimmed.

In some forms, a fistula graft device incorporates an effective amount of one or more antimicrobial agents or agents otherwise useful to inhibit the population of the device or surrounding tissue with bacteria or other deleterious microorganisms. Illustrative such agents can include, for example, silver compounds, such as silver salts (e.g. silver sulfate), dextran, chitosan, chlorhexidine, and/or nitric oxide donor compounds. In illustrative embodiments, such agents can be incorporated throughout the graft devices and/or on surfaces and/or selected regions thereof. These or other similar therapeutic agents can be incorporated directly on or in the implant constructs of the invention, or they can be incorporated with a suitable binder or carrier material, including for instance hydrogel materials.

Additional embodiments of the invention provide methods for treating fistulas that involve the use of flowable remodelable extracellular matrix material. In such embodiments, the flowable material can be used to fill openings and/or tracts of fistulas, including anorectal or other alimentary fistulas, and promote tissue ingrowth to close the fistulas. In this regard, the flowable material can be delivered in any suitable fashion, including for example forcible ejection from cannulated members such as catheters, sheaths, or needles. Suitable flowable, remodelable ECM materials for use in this aspect of the invention can be prepared, for example, as described in U.S. Pat. Nos. 5,275,826 and 5,516,533 or in International Publication No. W02005020847 (Cook Biotech Incorporated) published Mar. 10, 2005, which are each hereby incorporated by reference in their entirety. Such flowable materials can include solubilized and/or particulate ECM components, and in preferred forms include ECM gels having suspended therein ECM particles, for example having an average particle size of about 50 microns to about 500 microns, more preferably about 100 microns to about 400 microns. The ECM particulate can be added in any suitable amount relative to the solubilized ECM components, with preferred ECM particulate to ECM solubilized component weight ratios (based on dry solids) being about 0.1:1 to about 200:1, more preferably in the range of 1:1 to about 100:1. The inclusion of such ECM particulates in the ultimate gel can serve to provide additional material that can function to provide bioactivity to the gel (e.g. itself including FGF-2 and/or other growth factors or bioactive substances as discussed herein) and/or serve as scaffolding material for tissue ingrowth. Flowable ECM materials can also be used in conjunction with graft body devices as described herein, or implant bodies having other constructions. Implanted bodies can, for example, be provided at one or more locations of the fistula, e.g. within the primary opening, and can act as a confining barrier to an amount of flowable ECM material introduced against the barrier and filling the tract of the fistula to promote healing.

Further, in some aspects, the invention provides fistula deployment systems useful for implanting fistula graft devices in openings anywhere on or within the body of a patient to block at least a portions of the openings, for example, to block at least the primary opening of a urethro-vaginal fistulae, vesico-vaginal fistulae, tracheo-esophageal fistulae, gastro-cutaneous fistulae, and any number of anorectal fistulae, such as recto-vaginal fistula, recto-vesical fistulae, recto-urethral fistulae, or recto-prostatic fistulae.

All publications and patent applications cited in this specification are herein incorporated by reference as if each individual publication or patent application were specifically and individually indicated to be incorporated by reference. Further, any theory, mechanism of operation, proof, or finding stated herein is meant to further enhance understanding of the present invention, and is not intended to limit the present invention in any way to such theory, mechanism of operation, proof, or finding. While the invention has been illustrated and described in detail in the drawings and foregoing description, the same is to be considered as illustrative and not restrictive in character, it being understood that only selected embodiments have been shown and described and that all equivalents, changes, and modifications that come within the spirit of the inventions as defined herein or by the following claims are desired to be protected.

What is claimed is:

1. A fistula graft deployment system useful to treat a fistula having at least a primary opening in the alimentary canal, a fistula tract, and a secondary opening, the system comprising:
an elongate probing member having a lumen, said probing member including an end configured to pass through at least the secondary opening and a segment of the fistula tract;
a fistula graft device retaining element extending through said probing member lumen; and
a fistula graft device releasably retained by said retaining element, said fistula graft device including a biocompatible graft body configured to block at least the primary opening of the fistula.

2. The deployment system of claim 1, wherein said biocompatible graft body comprises a resorbable material.

3. The deployment system of claim 1, wherein said biocompatible graft body comprises a collagenous material.

4. The deployment system of claim 1, wherein said biocompatible graft body comprises a remodelable material.

5. The deployment system of claim 1, wherein said biocompatible graft body comprises an extracellular matrix material.

6. The deployment system of claim 5, wherein said extracellular matrix material comprises submucosa.

7. The deployment system of claim 6, wherein said submucosa comprises porcine submucosa.

8. The deployment system of claim 6, wherein said submucosa comprises small intestine submucosa, urinary bladder submucosa, or stomach submucosa.

9. The deployment system of claim 5, wherein said extracellular matrix material comprises serosa, pericardium, dura mater, peritoneum, or dermal collagen.

10. The deployment system of claim 1, wherein said biocompatible graft body comprises an expandable material.

11. The deployment system of claim 1, wherein said biocompatible graft body comprises at least one layer of compliant material.

12. The deployment system of claim 11, wherein said at least one layer of compliant material is deformable upon impingement by soft tissue surrounding the primary opening and is sized and shaped so as to be deformable to a three-dimensional volumetric body filling at least the primary opening of the fistula.

13. The deployment system of claim 1, wherein said graft body comprises two to ten layers of compliant material.

14. The deployment system of claim 13, wherein said two to ten layers of compliant material are bonded together.

15. The deployment system of claim 1, wherein said biocompatible graft body includes a rolled sheet material providing a volumetric body configured to fill at least the primary opening of the fistula.

16. The deployment system of claim 15, wherein said rolled sheet material provides spiral layers.

17. The deployment system of claim 16, wherein said spiral layers are compressed and bonded so as to form a substantially unitary structure.

18. The deployment system of claim 1, wherein said biocompatible graft body has a cross sectional dimension of from 3 mm to 20 mm.

19. The deployment system of claim 1, wherein said biocompatible graft body has a cross sectional dimension of from 5 mm to 15 mm.

20. The deployment system of claim 1, wherein said biocompatible graft body includes a tapered portion.

21. The deployment system of claim 1, wherein said probing member includes a flexible portion.

22. The deployment system of claim 1, wherein said retaining element is coupled to said probing member.

23. The deployment system of claim 1, wherein said retaining element is translatable along said probing member lumen.

24. The deployment system of claim 1, wherein said retaining element is slidably received within said probing member lumen.

25. The deployment system of claim 23, wherein said retaining element includes a deformable portion deformable upon sliding through said probing member lumen.

26. The deployment system of claim 25, wherein said deformable portion includes a piece of resilient wire having a generally closed circumference.

27. The deployment system of claim 25, wherein said deformable portion includes a curvilinear segment.

28. The deployment system of claim 1, wherein said probing member lumen provides a generally cylindrical space.

29. The deployment system of claim 1, wherein said probing member lumen provides a non-cylindrical space.

30. The deployment system of claim 29, wherein said non-cylindrical space includes a curvilinear lumen wall portion.

31. The deployment system of claim 29, wherein said non-cylindrical space includes a rectilinear lumen wall portion.

32. The deployment system of claim 1, wherein said probing member includes an opening in a side wall thereof.

33. The deployment system of claim 1, wherein said fistula graft device is releasably retained by said retaining element at said graft body.

34. The deployment system of claim 1, wherein said fistula graft device includes a suture in association with said graft body.

35. The deployment system of claim 34, wherein said fistula graft device is releasably retained by said retaining element at a point along said suture.

36. A method of deploying a fistula graft within a patient to treat a fistula having at least a primary opening in the alimentary canal, a fistula tract, and a secondary opening, the method comprising:
providing a fistula graft deployment system, the system comprising:
an elongate probing member having a lumen, wherein a portion of said probing member is positioned within the fistula tract;
a fistula graft device retaining element extending through the lumen of said probing member; and
a fistula graft device releasably retained by said retaining element, said fistula graft device including a biocompatible graft body;
manipulating said deployment system so as to lodge said graft body within the primary opening; and
releasing said fistula graft device from said retaining element.

37. The method of claim 36, wherein said manipulating includes moving said retaining element away from the primary opening inside the fistula tract.

38. The method of claim 36, wherein said biocompatible graft body is lodged so as to substantially seal the primary opening.

39. The method of claim 36, wherein said biocompatible graft body comprises a remodelable extracellular matrix material.

40. The method of claim 39, wherein said remodelable extracellular matrix material comprises submucosa.

41. The method of claim 39, wherein said remodelable extracellular matrix material comprises porcine submucosa.

42. A medical product useful to treat a fistula having at least a primary opening in the alimentary canal, a fistula tract, and a secondary opening, the product comprising:
an elongate probing member having a lumen, said probing member including an end configured to pass through at least the secondary opening and a segment of the fistula tract;
a fistula graft device retaining element extending through said probing member lumen;
a fistula graft device releasably retainable by said retaining element, said fistula graft device including a biocompatible graft body configured to block at least the primary opening of the fistula; and
a sealed package enclosing said elongate probing member, said fistula graft device retaining element, and said fistula graft device.
